(19) United States
(12) Patent Application Publication
(10) Pub. No.: US 2016/0354600 A1
(43) Pub. Date: Dec. 8, 2016

(54) Implantable Electrode Having An Adhesion-Enhancing Surface Structure

(71) Applicant: BIOTRONIK SE & Co. KG, Berlin (DE)

(72) Inventors: Gernot Kolberg, Berlin (DE); Michael Friedrich, Kleinmachnow (DE)

(21) Appl. No.: 15/168,922

(22) Filed: May 31, 2016

(30) Foreign Application Priority Data
Jun. 2, 2015 (DE) 10 2015 108 670.0
Jun. 2, 2015 (DE) 10 2015 108 671.9
Jun. 2, 2015 (DE) 10 2015 108 672.7

(51) Int. Cl. A61N 1/05

(52) U.S. Cl. CPC A61N 1/059; A61N 1/0573

(57) ABSTRACT

An electrode having an adhesion-enhancing surface structure.

### CROSS-REFERENCE TO RELATED APPLICATIONS

This patent application claims the priority of co-pending German Patent Application Nos. DE 10 2015 108 671.9; DE 10 2015 108 670.0; and DE 10 2015 108 672.7, all filed on Jun. 02, 2015 in the German Patent Office, the disclosures of which are hereby incorporated by reference in their entireties.

### TECHNICAL FIELD

The present invention relates to an implantable electrode.

### BACKGROUND

Implantable electrodes for use in or on the heart have been developed in conjunction with implantable cardiac pacemakers and have long been known in a large number of variants. By far, the greatest importance is attributed here to intracardially placed electrode leads, which are guided directly into the heart via a transvenous access point. Different types of fixing to the inner wall of the heart or in the trabecular meshwork of the ventricle have been proposed and also implemented in practice for these electrodes.

These electrodes also include different types of screw-in electrodes, which carry a fixing screw at the distal end. In addition, there are also intracardiac electrodes having a barb or fin arrangement for atraumatic fixing in the trabecular meshwork. Specially curved and/or branched electrode leads are also known, with which the pre-shaped basic form is intended to ensure a reliable bearing against the heart wall and, therefore, a secure transmission of stimulation pulses of the pacemaker thereto.

Whereas only intracardiac electrodes are essentially used for permanent use for pulse transmission of fixedly implanted pacemakers, epicardiac electrodes are used above all for the temporary stimulation of the heart during or following surgical interventions. Furthermore, they are used in the form of large-area surface electrodes (patch electrodes) in conjunction with implantable defibrillators.

In the meantime, compact pacemakers have been developed, with which the electrically active area of the electrode sits directly on the housing body, i.e., no electrode lead is provided to the electrode head (also referred to as leadless pacemakers).

A therapeutic or diagnostic device intended to be effective at a specific location must be fixed there so as to retain its position in the event of movements. This is often imperative in order to be able to maintain the therapeutic effect. A stimulation electrode, for example, targets a cell area in the heart carefully selected by a doctor. The electrical parameters for the therapy are set for this area. If the position of the electrode changes, the therapeutic effect will most likely be lost, because on the one hand the parameters are unsuitable for the new position, or on the other hand because the area does not support the therapeutic effect. In the worst-case scenario, the patient may even be put at risk, because stimulation of an incorrect area can lead to dangerous effects.

Another reason to fix components in the vascular system or in or on the heart is to hold the components in a secure position. Otherwise, the components would be swept along by the blood flow or, as a result of gravity, would reach locations where they might be dangerous for the patient. They might thus block vessels, resulting in embolisms, heart attack or stroke.

A reliable fixing of the electrodes at the implantation site is therefore vital for diagnostic and therapeutic purposes. In the event of a dislodgement of the electrodes, the desired function can no longer be ensured, and significant complications could occur. The fixing mechanism itself should have a minimal effect on the organism. A purely mechanical fixing by sewing, or using anchoring structures or clamping elements might damage the affected tissue in a lasting manner and potentially irreparably. Adhesion-enhancing glues can lead to incompatibility reactions, and electrodes fixed using such glues generally can no longer be separated from the adhering tissue without damage.

The cited solutions therefore often result in damage to the tissue structures. This damage initializes connective tissue proliferation, which positively assists the fixing. A disadvantage of the connective tissue, however, is the change of cell structures. This change can be detrimental to the therapeutic effect, for example, as a result of an increase in the stimulus threshold in the event of stimulation. The sewing of the components is very secure, but is associated with great effort. An epicardiac electrode can actually be sewn in place only if the ribcage is opened. By contrast, screwing-in using a helical needle or support against the vessel walls is accompanied again and again by dislodgements.

The discussed problems of the prior art can be solved or at least mitigated with the aid of the implantable electrode according to the invention for use in or on the heart. The electrode is characterized in that the electrode comprises an electrode head having an adhesion-enhancing surface structure, preferably a gecko structure.

The present invention is directed toward overcoming one or more of the above-mentioned problems.

### SUMMARY

The present invention thus utilizes an alternative possibility for the connection of different surfaces via the phenomenon of dry adhesivity. Dry adhesivity is understood in the present case to mean the formation of adhesive forces between surfaces without adhesion-enhancing substances, such as, for example, glues. Adhesion systems of this type are also known, for example, from nature, for example in the case of gecko legs or insect legs. It is assumed that in such systems the adhesive forces are based on van-der-Waals forces. The adhesion-generating surface for this purpose has an adhesion-enhancing surface structure, for example, a multiplicity of brush-like or hair-like elements, which lead to a very large increase in the available contact area. With the enlargement of the contact area, the strength of the adhesion forces formed in the event of contact consequently also increases. The use of adhesion-enhancing surface structures of this type for attachment to tissue is proposed, for example, by Alborz Mandavi et al., ‘A Biodegradable and Biocompatible Gecko-Inspired Tissue Adhesive’, PNAS (2008), Vol. 105, No. 7, 2307-2312.

In the field of heart electrodes, damage to the tissue, as occurs in the case of conventional methods (e.g., sewing or screwing in) and can lead to a weakening of the therapeutically usable tissue areas, can be avoided with the aid of the adhesion-enhancing surface structure. The avoidance of damage, for example, also makes a minimally invasive epicardiac application safe, because coronary arteries can no longer be accidentally damaged during the fixing. Furthermore, a very quick fixing is possible by lightly pressing on the component at the desired point, such that new implantation methods can be developed. Nowadays, the greatest outlay involved with the implantation lies in the fixing of the components. Part of the implantation diameter must nowadays be allocated to the fixing tools. Particularly in the case of epicardial application, a large opening in the ribcage is necessary in order to sew on or screw in the electrodes. Conventional intracardiac screw electrodes having an actively retractable screw are also technically complex and require a stable and large internal helix so as to be able to transmit the torsion. With the present invention, it is possible to dispense with the complex and high-risk mechanism. Lastly, the adhesion-enhancing surface structure enables the implant to be detached again in a simple and planned manner. In spite of a good fixing, a changeover of the component to be fixed is possible, wherein the fixing can also be easily detached again without detaching unintentionally.

The adhesion-enhancing surface structure may have between 10 and 1,000,000 rods per square millimeter, for example. The ratio of diameter and length of the rods may be between 1:2 and 1:2,000. The cross section of the rod may be cross-profiled, for example, completely or partially round, triangular, rectangular, square or internally hollow. It may have a T-profile or may correspond to a crescent-shaped outline. A preferred bending direction of the rod can thus be predefined. Alternatively, or in combination, the rods can be pre-bent or obliquely attached. A uniform bending direction of the rods may prevent the rods from becoming entangled with one another. The rods may also have a longitudinal profile. They may thus be thickened at the root, where they bear against the component to be fixed, and may taper toward the end.

The adhesion-enhancing surface structure can also consist of rods that branch out. The end of the last branch can be thickened again. The greatest extent of the thickened portion corresponds at most to 100 times the rod diameter on which the thickened portion sits. The end of the last branch may also be planar or rounded or pointed. A lobe-like structure, similarly to a scoop, can be located at the end of the last branch and is attached at one end. The lobe-like structure is preferably attached at one end to the rods in such a way that the angle of the rods is continued. In the event of a transverse force of the component in the detaching direction (for example, in an anticlockwise direction), the lobe-like structure peels away from the tissue, which significantly facilitates the detachment, whereas in the event of transverse force in the other direction only a shear force is caused, which not only does not detach the fixing, but aids the fixing.

The fixing and detachment forces can be set by organization of the bending direction of the rods on the surface. The structures are fixed particularly well when as many rods as possible absorb the tensile forces simultaneously. If the fixing is to be released, the rods must be individually loaded, where possible, so as to enable a detachment even with low forces. Due to the preferred bending direction of the rods, a force acting laterally on the component can be converted into a tensile force or into a compressive force, depending on direction. A force against the rod orientation leads to a force compressing the rod, which causes the rod to bend, as a result of which a rolling motion occurs at the fixing surface, which peels off the fixing surface. This effect can also be utilized over a number of rod sections. For example, only the lower end of the rods may thus be provided with a preferred direction. The subsequent, for example, branched structures are peeled off. An equivalent effect is attained when the rods do not have a preferred bending direction, but are already obliquely attached or pre-curved.

A special embodiment of the rods, which are pre-bent or provided with a preferred bending direction, is one in which the rods are pre-bent about a pivot point, preferably the point of the electrically active or sensitive area in one direction, preferably in an anticlockwise direction. A rotation at the component in an anticlockwise direction rolls each individual rod end about the fixing point and peels it off. The fixing can thus be provided by pressing the component on or by rotation in a clockwise direction. Detachment occurs by rotation in an anticlockwise direction.

Besides the specified tangential orientation of the rods, further structured arrangements are conceivable, for example, an area in which the rods point in one direction is detachable by a force in this direction and is stable in the other direction.

If the component is to be detached by means of an orthogonally acting force, it is expedient for the fixing area to be designed as a membrane and for the rods to point towards the center point of the membrane. If the component is removed perpendicularly, the rods detach from the outside in. This process can be triggered alternatively by a ram, which presses from the inside onto the membrane, or by fluid pressure.

The adhesion-enhancing surface structure can be manufactured in principle from any material that can be connected to the further constituents of the electrode and that is sufficiently compatible for an intracorporeal use. The adhesion-enhancing surface structures preferably consist of a polymer material, in particular, a silicone. Further possible materials for the structures include, for example, carbon materials, in particular in the form of fibers and nanotubes, polypropylene, polytetrafluoroethylene (PTFE), ethylene tetrafluoroethylene (ETFE), polycarbonate, polystyrene, polylactides, for example, PDLLA, synthetic spider silk, polyurethanes and copolymers thereof, polyimide, polyamide, polyether ether ketone (PEEK), polysulfone, polyethylene, polyoxymethylene (POM), polyether block amide, chitin, collagen, cellulose, keratin, metals, glass, and ceramic. The adhesion-enhancing surface structures may consist, in particular, of an electrically conductive material so as to also enable electrical contact in addition to the mechanically stable contact. This structure can be coated by a suitable substance, such as poly(dopamine methacrylate-co-2-methoxyethyl acrylate) (p(DMA-co-MEA)), so as to improve the adhesive strength in liquid media, or with steroids, so as to suppress inflammation processes. Substances that promote ingrowth behavior can also be used.

An adhesion-enhancing surface structure can be produced by different methods. By way of example, negative molds can be produced by lithographic methods, such as electron beam lithography and laser lithography, or by etching methods. In a subsequent casting method, the positive surface with hair-like extensions is then produced starting from the negative mold (for example, see A.K. Geim et al., Nature Mater. 2, 461-463 (2003) and H. Lee, B.P. Lee and P.B. Messersmith, Nature 448, 338341 (2007)).

The adhesion-enhancing surface structure is preferably arranged on an end face of the electrode head of the electrode. A dislodgement of the electrically active areas, which serve to stimulate the adjacent tissue or to detect electrophysiological processes, is effectively prevented as a result. The electrode head is pressed easily against the tissue at the intended position, and the adhesion-enhancing surface structure holds the head in the desired position.

In accordance with a further, preferred variant of the previous embodiment, the electrode head widens in a plate-like manner starting from a distal end of the electrode lead, and the region of the electrode head widened in a plate-like manner can be reversibly folded in the direction of the electrode lead. The electrode head thus has a sort of peripheral lamella, which can be folded in the proximal direction of the electrode lead and can then be laid again in the original position or can reset itself. This can be achieved, for example, in that at least part of the electrode head protruding beyond the cross section of the electrode lead is formed from a material having elastic properties, for example, a polymer. Due to the special shaping of the electrode head, the adhesion-enhancing surface structure and, therefore, potential contact area relative to the adjacent tissue can be enlarged. During the minimally invasive implantation, however, the regions of the electrode head widened in a plate-like manner bear against the electrode lead and are held there for example in a suitable sleeve, such that the cross section remains sufficiently small. Only at the implantation site is the head electrode expanded again, for example by retracting the aforementioned sleeve. The use of an elastic material additionally enables an improved fit of the adhesion-enhancing surface structure to the tissue, which further increases the adhesion forces.

In a development of the aforementioned embodiment, spacers are arranged on the end face of the electrode head and protrude beyond the adhesion-enhancing surface structure. In this way, the adhesion-enhancing surface structure can be prevented from coming into contact with the inner face of a sleeve, which holds the folded region of the electrode head widened in a plate-like manner in position as the electrode head is guided to the implantation site. The spacers are thus dimensioned such that the adhesion-enhancing surface structures located on the end face cannot develop any adhesion relative to the inner face of the sleeve.

Alternatively, the adhesion-enhancing surface structure can be designed such that a force acting radially outwardly from the center point of the electrode head counteracts an adhesion of this surface structure to an adjacent surface. In other words, the adhesion-enhancing surface structure can be fashioned such that a sliding along the inner face of the aforementioned sleeve in the distal direction is possible. This can be achieved, by way of example, in such a way that the structure has a multiplicity of rods, of which the ends are bent toward the end face of the head electrode, such that they are inclined toward the middle of the end face. Further possibilities for organizing the bending direction of the rods have already been described previously.

When the electrode head has a fixing screw centrally, a gradual unscrewing of the screw as a result of the constant tissue movement can be prevented in accordance with the same principle. The adhesion-enhancing surface structure is then designed such that a force acting with the thread direction of the fixing screw counteracts an adhesion of the surface structure to an adjacent surface. In other words, the screw can be screwed in unhindered as far as the desired depth, because the adhesion between the adhesion-enhancing surface structure and the adjacent tissue is detached again and again by the specific shaping of the structure. However, a rotation in the opposite direction is opposed by the full adhesion force of the structure.

The discussed prior art problems can also be solved or at least mitigated with the aid of the implantable electrode according to the present invention, and with an elongate electrode lead and an electrode head arranged distally thereon. The electrode is characterized in that the electrode lead has an adhesion-enhancing surface structure, preferably a gecko structure. The structures are thus disposed laterally on the electrode lead and enable a fixing in vessels, for example, the coronary arteries, or a fixing to the heart wall (endocardially or epicardially).

In accordance with a further embodiment of the aforementioned electrode, the adhesion-enhancing surface structure is arranged in the region of a pre-shaping of the electrode lead that serves to be supported against a vessel wall. The positioning of specially curved and/or branched electrode leads is thus assisted in that the pre-shaped basic form has an adhesion-enhancing surface structure in predefined regions. A particularly reliable bearing against the heart wall is ensured as a result.

The implantable electrode is preferably a heart electrode, for example, a (possibly also leadless) pacemaker or defibrillator. However, the electrode is not necessarily limited to this field of application and for example can also be used in implants for diagnostic or therapeutic treatment of the urethra, the bladder, in the mouth, nose or oesophagus, in the digestive system, in the ear canal, or uterus.

Further embodiments, features, aspects, objects, advantages, and possible applications of the present invention could be learned from the following description, in combination with the Figures, and the appended claims.

Further preferred embodiments of the present invention will emerge from the dependent claims and the following description.

### DESCRIPTION OF THE DRAWINGS

The present invention will be explained hereinafter on the basis of an exemplary embodiment and associated drawings, in which:

FIG. 1 shows a schematic sectional view through an adhesion-enhancing surface structure of an electrode according to the present invention having a multiplicity of rods.

FIG. 2 shows exemplary cross sections of rods of the adhesion-enhancing surface structure.

FIG. 3 shows an illustration of the design possibilities for the rods of the adhesion-enhancing structure by branching of the rods and shaping in the region of the ends.

FIG. 4 shows a schematic illustration of a preferred bending direction of the rods of an adhesion-enhancing surface enabling a fixing in the event of rotation in a clockwise direction and detachment in the event of rotation in an anticlockwise direction.

FIG. 5 shows a schematic illustration of a preferred bending direction of the rods of an adhesion-enhancing surface enabling a detachment by orthogonally acting forces.

FIG. 6 shows a schematic illustration of a preferred bending direction of the rods of an adhesion-enhancing surface enabling a displacement in one direction.

FIG. 7 shows a heart electrode with fixing screw and an adhesion-enhancing surface.

FIG. 8 shows an epicardiac heart electrode with adhesion-enhancing surface.

FIG. 9A-9C show a further embodiment of a heart electrode with fixing screw and an adhesion-enhancing surface in three different views.

FIG. 10A-10B show two further embodiments of heart electrodes having an adhesion-enhancing surface.

FIG. 11 shows an electrode lead having an adhesion-enhancing surface in a first embodiment.

FIG. 12 shows an electrode lead having an adhesion-enhancing surface in a second embodiment.

### DETAILED DESCRIPTION

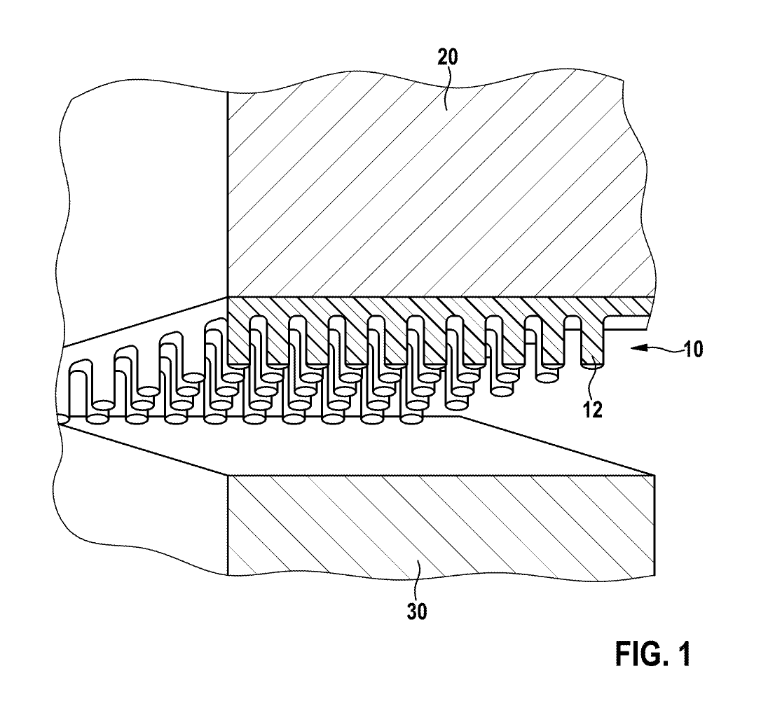

FIG. 1 shows a schematic sectional view through an adhesion-enhancing surface structure 10, which is arranged on an upper side of an electrode 20. The structure 10 has a multiplicity of rods 12, which protrude approximately perpendicularly from the upper side of the electrode 20. The surface modified by the structure 10 is disposed above a tissue surface 30, onto which said modified surface must be briefly pressed. The structure 10 preferably consists of an elastic material, for example, silicone. As said structure is pressed against the tissue surface, the rods 12 yield, so that the modified surface can be guided more closely against a surface of a tissue 30. In this way, a contact area between the rods 12 and the tissue 30 and, therefore, adhesion force between the components can be increased. When the pressure is removed, the rods 12 are no longer in contact with the surface of the tissue 30. Potential unevennesses are therefore compensated for by the elastic stretching of the rods 12.

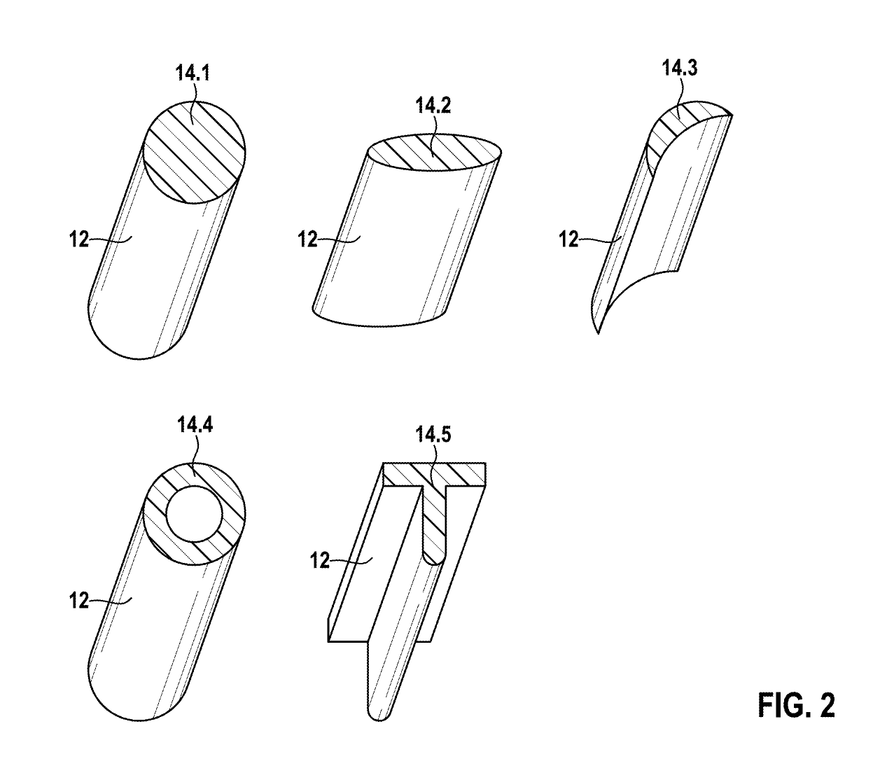

The rods 12 may have a different cross section in the longitudinal direction. FIG. 2, by way of example, shows five different cross sections of rods 12 and the influence thereof on the bending behavior. A round cross section 14.1 does not result in any preferred bending direction, but can be manufactured particularly easily. However, for example, a rod 12 can be provided so as to bend only in one plane under load (e.g., flat cross section 14.2), or the rod 12, in addition to bending in just one plane, can be provided so as to bend more easily in one direction than in the other direction (e.g., crescent cross section 14.3). A rod 14 can also have a cavity (e.g., cross section 14.4) or a T-profile (e.g., cross section 14.5). Both the bending behavior and the stretchability and compressibility can therefore change. A tubular rod thus has a flatter spring characteristic curve compared with a fully filled rod. This is expedient because greater height differences between component and tissue can be compensated for as a result. The objective is that all rods 12 transmit, where possible, the same force from the component of the tissue 30. In the case of a steep spring characteristic curve, a rod 12 that must compensate for a long path would transmit more force and therefore would pull away again more quickly as a result of the stretching.

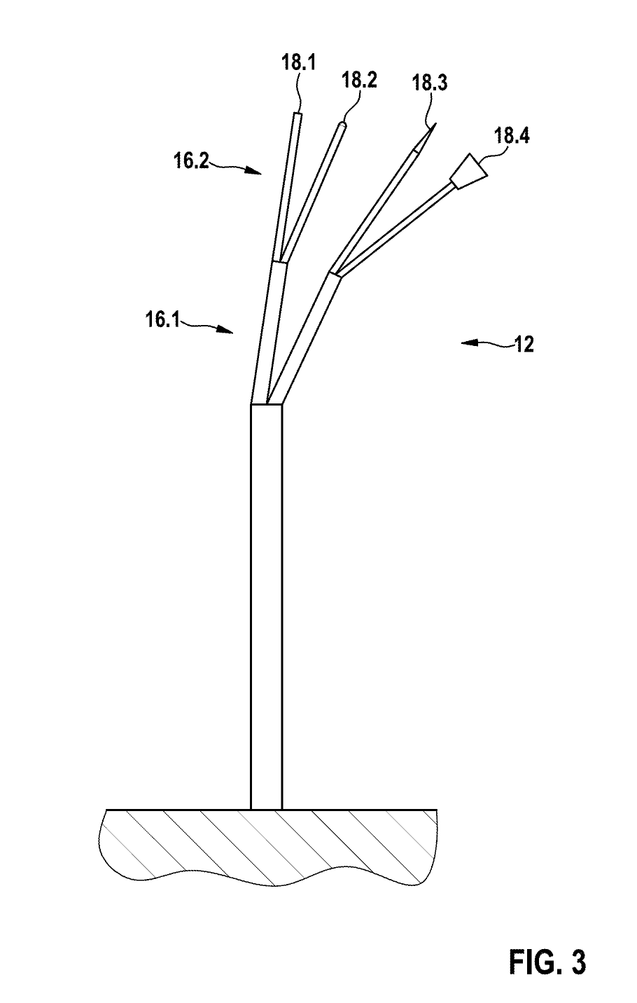

FIG. 3 illustrates purely schematically contact points for the specific optimization of the shape of the rods 12 of the adhesion-enhancing structure in the application in question. The forces between tissue 30 and component can be set via rod structures of this type. The rod 12 may thus have branches, here two additional branch sections 16.1 and 16.2 by way of example. For example, the primary rod portions can thus be rigid and long and, therefore, can compensate for large height differences; the rod portions of the first branch section 16.1 serving to compensate for medium height differences, whereas the rod portions of the second branch section 16.2 contact the tissue surface. Here, the rod portions preferably become shorter and more delicate from section to section.

The shaping in the region of the ends of the rods 12 can also vary. The end may be cut, for example, straight (tip 18.1), rounded (tip 18.2), pointed (tip 18.3) or lobe-like (tip 18.4). A particularly preferred embodiment is the lobe-like structure attached at one end (tip 18.4). The advantage of this embodiment is that the one-ended attachment facilitates the detachment, because a tensile force can thus be transferred into a peeling load. The high adhesion force is produced from the sum of the microscopic fixing surfaces. A detaching force must be very high, accordingly. When the force is transferred into a peeling load, however, the adhering structures are loaded one by one so heavily that this results in a detachment. The lobe-like structure can be rolled over again by the tissue 30.

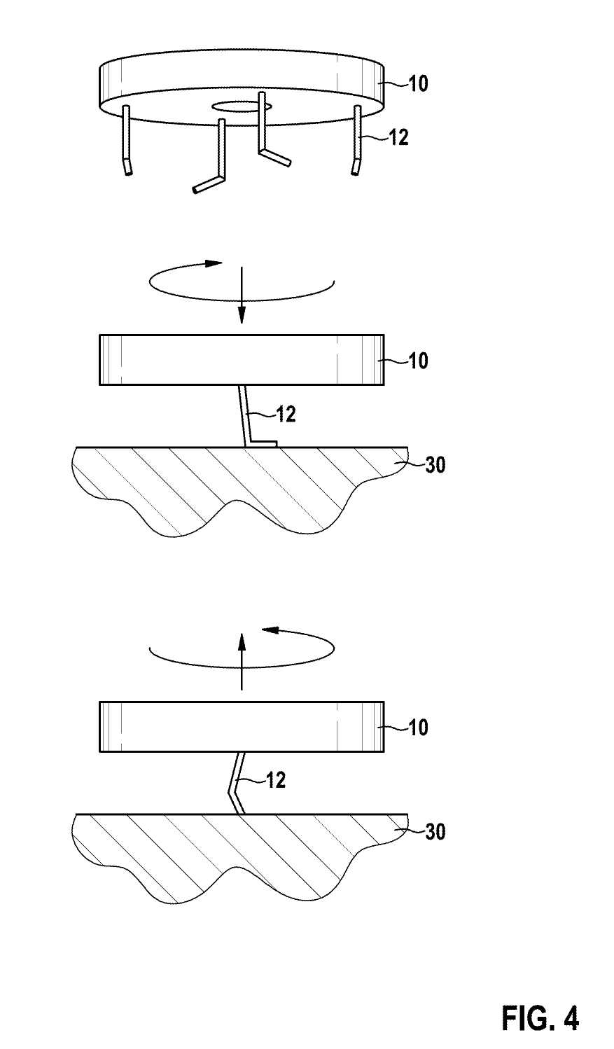

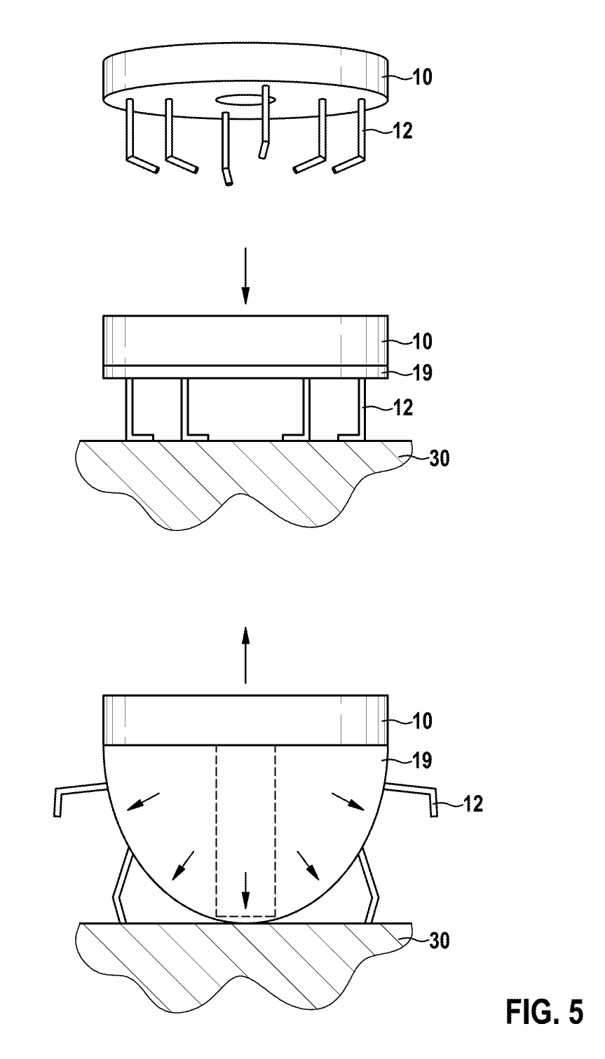

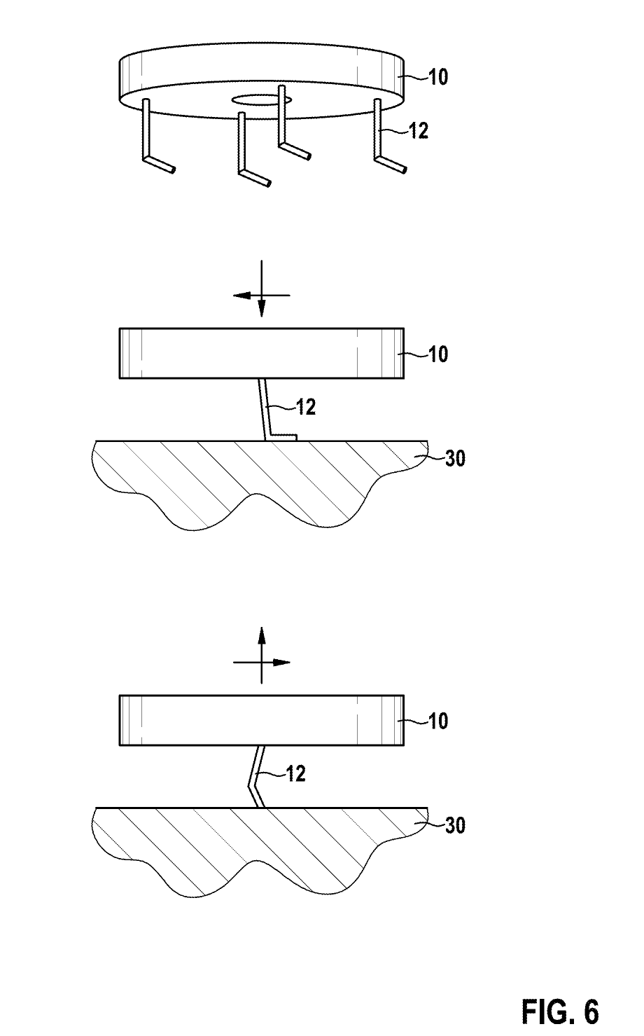

Various orientations of the rods 12 are illustrated in FIGS. 4 to 6, by means of which a component can be detached again from the tissue 30 by a defined movement. A schematic illustration of a preferred bending direction of the rods 12 of the adhesion-enhancing surface 10, which enables a fixing in the event of rotation in a clockwise direction and a detachment in the event of rotation in an anticlockwise direction, can be inferred from FIG. 4. A preferred bending direction of the rods 12 of the adhesion-enhancing surface 10 can alternatively also be predefined such that a detachment is enabled by orthogonally acting forces (see FIG. 5). For this purpose, the rods 12 can be arranged on an elastic membrane 19, on the rear side of which pressure is exerted, for example, using a ram or by entry of a medium for detachment. By means of an appropriate specification of the preferred bending direction of the rods 12 of the adhesion-enhancing surface 10, a displacement in one direction can also be made possible (see FIG. 6).

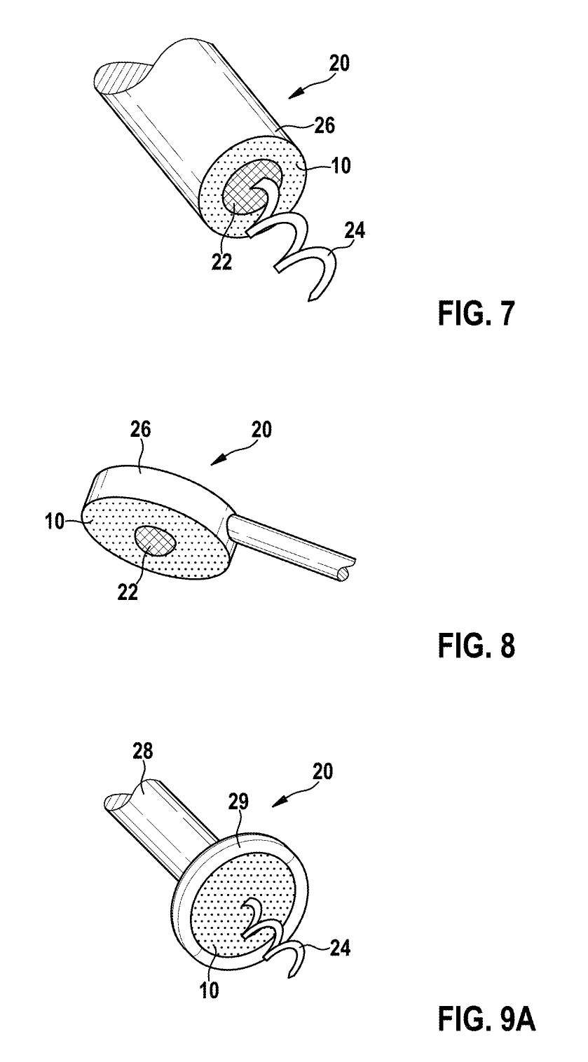

FIG. 7 shows a tip of a heart electrode 20 of a conventional pacemaker, or also leadless pacemaker. Besides an electrically active surface 22, a head 26 of the electrode 20 has a fixing screw 24 arranged centrally on the end face for mechanical anchoring in the epicardium. The adhesion-enhancing surface structure 10 is disposed around the fixing screw 24 and is designed such that it prevents an independent rotation of the body (see the embodiment according to FIG. 4 in this respect).

FIG. 8 shows the tip of an epicardial heart electrode 20. The adhesion-enhancing surface 10 is again arranged on the electrode head 26 around the electrically active surface 22. The electrode 20 is pressed easily from the outside against the heart and is fixed independently by the fixing surface, without damaging the tissue.

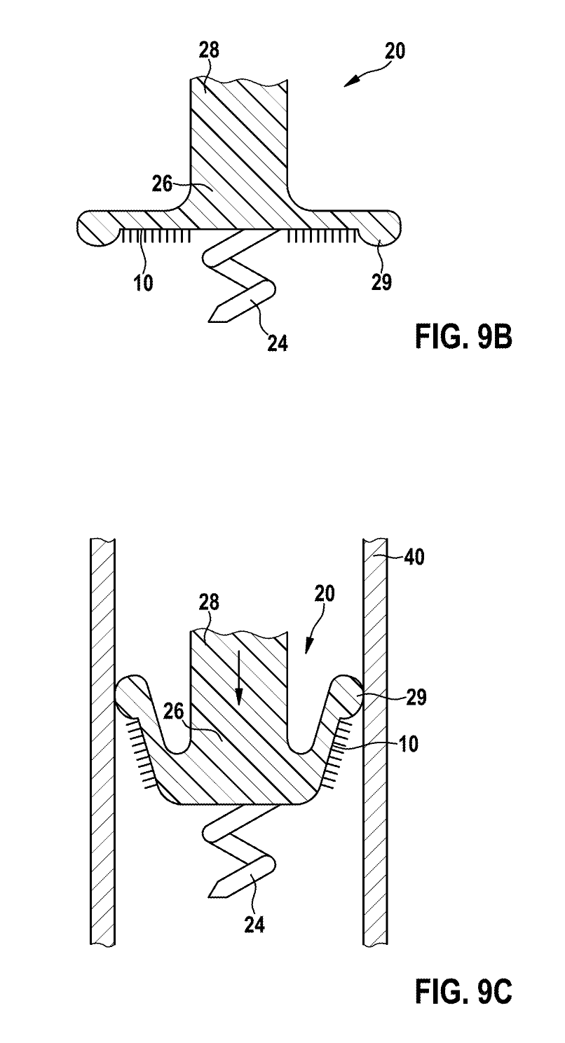

FIGS. 9A-9C show a further exemplary embodiment of a heart electrode 20 with fixing screw 24 and adhesion-enhancing surface 10 in three different views. The electrode head 26 widens in a plate-like manner starting from a distal end of an electrode lead 28. The region of the electrode head 26 widened in a plate-like manner can be reversibly folded in the direction of the electrode lead 28 and for this purpose consists of an elastic material. The outer edge of the end face of the electrode head 26 is reinforced and acts as a spacer 29 when the folded electrode head 26 is disposed in an insertion instrument 40 (see FIG. 9C).

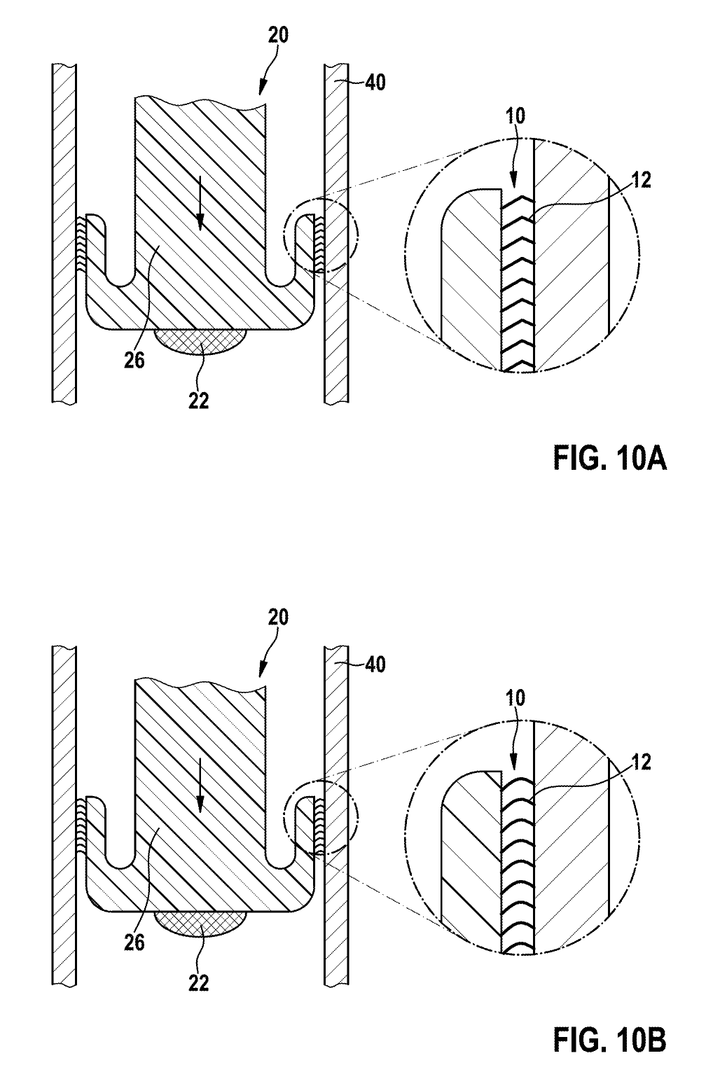

FIGS. 10A-10B show two further exemplary embodiments of heart electrodes 20 having an adhesion-enhancing surface 10. The electrode 20 has a centrally arranged electrically active region 22, which sits on an electrode head 26 widened in a plate-like manner. By purposeful orientation of the rods 12 forming the adhesion-enhancing surface structure 10, a displacement of the head 26 in the insertion instrument 40 in one direction is possible. The adhesion-enhancing surface structure 10 is thus designed such that a force acting radially outwardly from the center point of the electrode head 26 counteracts an adhesion of the surface structure 10 to an adjacent surface.

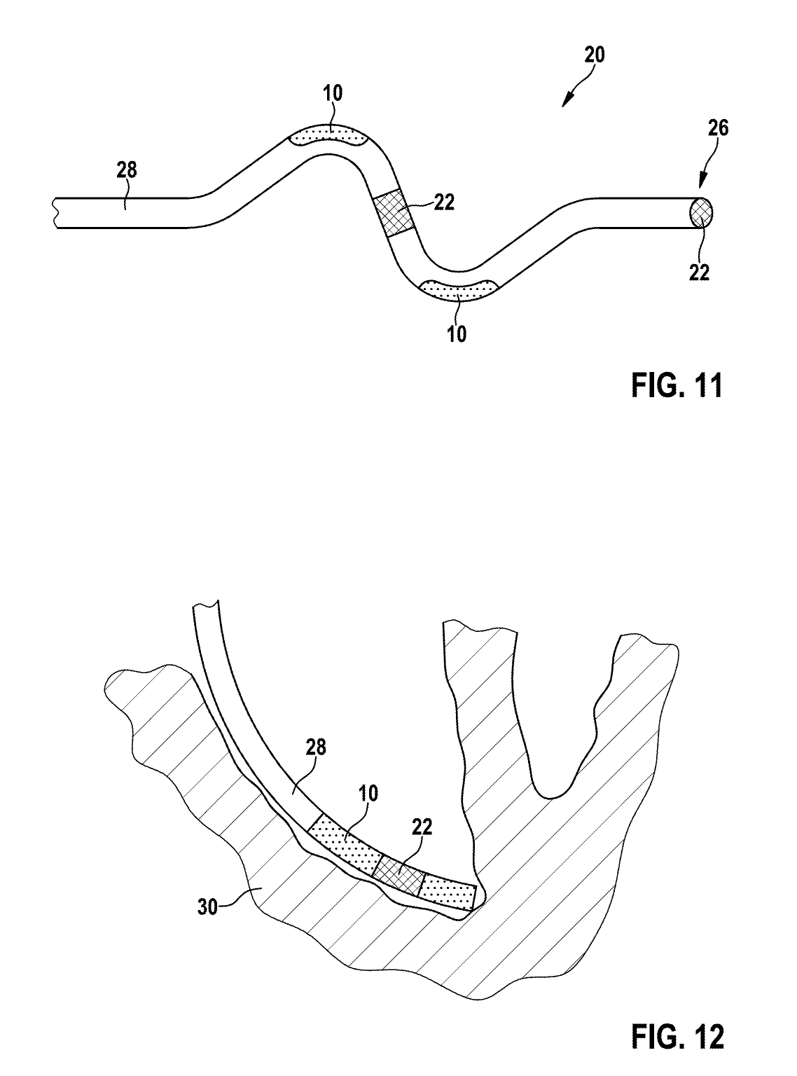

FIG. 11 shows an electrode 20 having an elongate electrode lead 28 and an electrode head 26 arranged distally thereon. The adhesion-enhancing surface structure 10 is provided here in two different portions of the electrode lead 26. Specifically, the adhesion-enhancing surface structures 10 are disposed in the region of a deformation of the electrode lead 28 used for support against a vessel wall. The positioning of specially curved and/or branched electrode leads 28 is thus assisted in that the pre-shaped basic form has an adhesion-enhancing surface structure 10 in predefined regions. A particularly reliable bearing against the heart wall is ensured as a result.

FIG. 12 shows a further exemplary embodiment of the electrode lead 28 having an adhesion-enhancing surface 10. The illustrated intracardiac heart electrode 20 has radially arranged fixing areas. This illustration shows an actively fixable electrode 20, however, a passively fixable atraumatic embodiment is also conceivable.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. An implantable electrode, wherein the electrode comprises an electrode head having an adhesion-enhancing surface structure.

2. The electrode according to claim 1, wherein the electrode head widens in a plate-like manner starting from a distal end of an electrode lead, and the region of the electrode head widened in a plate-like manner can be reversibly folded in the direction of the electrode lead.

3. The electrode according to claim 2, wherein spacers are arranged on the end face of the electrode head, which protrude beyond the adhesion-enhancing surface structure.

4. The electrode according to claim 3, wherein the adhesion-enhancing surface structure is designed such that a force acting radially outwardly from the center point of the electrode head counteracts an adhesion of the surface structure to an adjacent surface.

5. The electrode according to claim 2, wherein the electrode head has a fixing screw centrally and the adhesion-enhancing surface structure is designed such that a force acting with the thread direction of the fixing screw counteracts an adhesion of the surface structure to an adjacent surface.

6. The electrode according to one claim 1, wherein the adhesion-enhancing surface structure is formed from a polymer material.

7. The electrode according to claim 6, wherein the polymer material is a silicone.

8. The electrode according to claim 1, wherein the adhesion-enhancing surface structure is a gecko structure.

9. An implantable electrode having an elongate electrode lead and an electrode head arranged distally thereon, wherein the electrode lead has an adhesion-enhancing surface structure.

10. The electrode according to claim 9, wherein the adhesion-enhancing surface structure is arranged in the region of a pre-shaping of the electrode lead used for support against a vessel wall.

11. The electrode according to claim 9, wherein the adhesion-enhancing surface structure is formed from a polymer material.

12. The electrode according to claim 11, wherein the polymer material is a silicone.

13. The electrode according to claim 9, wherein the adhesion-enhancing surface structure is a gecko structure.
